(19) United States
(12) Patent Application Publication
(10) Pub. No.: US 2009/0162308 A1
(43) Pub. Date: Jun. 25, 2009

(54) USE OF 2,4'-DIMETHYLPROPIOPHENONE AS A FRAGRANCE SUBSTANCE

(75) Inventors: Walter Kuhn, Holzminden (DE); Stefan Lambrecht, Hehlen (DE); Johannes Panten, Hoxter (DE); Wilhelm Wiedmann, Bevern (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/337,751

(22) Filed: Dec. 18, 2008

(30) Foreign Application Priority Data
Dec. 19, 2007 (EP) 07 123 731.7

(51) Int. Cl. A61K 8/49; A61K 8/33; C11D 3/50; A61K 47/08; A61L 9/01; A61Q 13/00; C11C 5/00; C10L 1/18; C09G 1/00

(52) U.S. Cl. 424/65; 512/21; 512/11; 510/102; 514/772; 44/437; 44/275; 106/3; 512/4

(57) ABSTRACT

The use of 2,4′-dimethylpropiophenone as a fragrance substance is described, preferably as a fragrance substance for imparting, modifying and/or reinforcing one, two, three or all the odor notes floral, rosy, rose oxide-like and herbal-like, preferably both of the odor notes rose oxide-like and herbal-like.

### CROSS-REFERENCE TO RELATED APPLICATION

This application claims benefit of priority to EP 07 123 731.7, filed on Dec. 19, 2007, which is incorporated herein by reference in its entirety.

The present invention relates to the use of 2,4′-dimethylpropiophenone as a fragrance substance, above all for imparting, modifying and/or reinforcing the odor notes floral, rosy, rose oxide-like and/or herbal-like. A further aspect relates to fragrance substance compositions, which comprise an effective quantity of 2,4′-dimethylpropiophenone as well as one or more further fragrance substances, preferably fragrance substances of the fougère type or the rose type, as well as corresponding methods for producing these fragrance substance compositions.

A further aspect of the present invention relates to perfumed articles which contain a corresponding fragrance substance composition.

The invention additionally relates to the use of 2,4′-dimethylpropiophenone or corresponding fragrance substance compositions for providing (a) hair or (b) textile fibres with a floral, rosy, rose oxide-like and/or herbal-like odor as well as corresponding methods.

The invention also relates to certain uses of 2,4′-dimethylpropiophenone as a means for increasing the substantivity and/or retention of a fragrance substance composition, the use as means for increasing the complex stability of a fragrance substance composition, the use as means for changing, masking and/or reducing a medical odor note in a fragrance substance composition as well as the use as a means for increasing the odor of other fragrance substances perceived over a surfactant-containing aqueous solution.

See below with regard to further features of the fragrance substance compositions, perfumed articles, methods and uses according to the invention.

Despite a large number of already existing fragrance substances, there is still a general need in the perfume industry for new fragrance substances which have additional positive secondary properties beyond their primary, namely odor, properties, such as, for example, higher stability under certain application conditions, a higher yield or a better adhesion capacity, or else lead to better sensory profiles owing to synergistic effects with other fragrance substances.

There is also basically a need in the perfume industry for further fragrance substances, which are suitable for the production of fragrance substance compositions or perfumed articles. In particular, there is a need for fragrance substances, which lead to an increased benefit in fragrance substance compositions owing to the technical properties mentioned above. Thus, for example, the use quantities and the number of fragrance substances in corresponding formulations may be optimized and/or minimized, for example, by the use of fragrance substances with a higher stability (particularly with respect to aggressive media such as hair colourants or bleaching agents) and this leads to lasting saving of resources in the perfuming of consumer items.

There is therefore a need, in particular, in the perfume industry for further fragrance substances with better sensory profiles and higher stability, above all with respect to aggressive media.

In this respect, there is a need, in particular, in the perfume industry for fragrance substances with floral, herbal (herbaceous) or herbal-like notes. This is to be taken to mean, in the framework of the present text, an odor which is similar to the odor of the naturally occurring rose oil or that of its constituents, but has an additional herbal note. The odor of rose oxide as a constituent of natural Bulgarian rose oil, for example, is described as follows in the literature: floral-geranium-like, green, hard (source: S. Arctander, Perfume and Flavor Chemicals, volume I and II, Montclair, N. J., 1969, self-published, number 2809). The perfume term “herbal” is more precisely described as “grass-green, spicy and medical” (source: P. M. Müller, D. Lamparsky, Perfumes—Art, Science and Technology, 1991, Elsevier Applied Science, London, New York, page 284).

The primary object of the present invention was to disclose a new rose fragrance substance with an additional herbal character, as well as corresponding fragrance substance compositions. Fragrance substances with a rose oxide-like and herbal-like odor note were preferably to be disclosed. Furthermore, these fragrance substances or the fragrance substance compositions were to have the aforementioned technical properties or advantages.

Furthermore, corresponding advantageous uses of the fragrance substance were to be provided by the present invention. Further objects, on which the present invention is based, emerge from the following embodiments and the accompanying patent claims.

In the scope of the objects according to the invention, the aim was above all to use corresponding fragrance substances in fragrance substance compositions of the fougère type.

The present invention relates, according to a primary aspect, to the use of 2,4′-dimethylpropiophenone as a fragrance substance. 2,4′-dimethylpropiophenone is particularly suitable for use as a fragrance substance for imparting, modifying and/or reinforcing one, two, three or all the odor notes floral, rosy, rose oxide-like and herbal-like, preferably both of the odor notes rose oxide-like and herbal-like.

The corresponding notes of an after-odor are also to be associated with the odor notes described in the scope of the present text. In other words, 2,4′-dimethylpropiophenone is suitable, in particular for use as a fragrance substance for imparting, modifying and/or reinforcing one, more or all the aforementioned odor notes and/or an after-odor with one or more of the aforementioned notes. The corresponding notes of an after-odor are likewise to be associated with the odor notes described in the further aspects, stated below, of the present invention.

As becomes clear with the aid of the following statements, it was particularly surprising that 2,4′-dimethylpropiophenone is suitable as a fragrance substance in the sense of the use(s) described above.

The structural formula of 2,4′-dimethylpropiophenone is reproduced below:

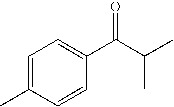

2,4′-dimethylpropiophenone (CAS number: 50390-51-7) is already known. The odor properties of 2,4′-dimethylpropiophenone have hitherto not been described, however.

In DE 2808817, new acyl-polyalkyl-indan compounds are described, as well as the use thereof as a basic substance for fragrance substances, corresponding fragrance substance compositions, perfumed substances and perfumed articles. Described there in the scope of a method for producing certain compounds is a ketone of a general structural formula, with 2,4′-dimethylpropiophenone coming under this structural formula, which is reduced in a known manner to the corresponding alcohol, in order to then be converted in a plurality of steps to form the desired polyalkyl-indan. A musk character is described for the synthesised perfume substances.

DE 10236918 also only describes a ketone of a general structural formula as the starting substance for the synthesis of corresponding fragrance substances, 2,4′-dimethylpropiophenone coming under this structural formula. In this case, a method is disclosed for producing alkylphenylcarbinols, corresponding alkylphenyl ketones being hydrogenated in the presence of nickel catalysts and alkaline additives to produce the desired alkylphenylcarbinols.

A novel method for preparing substituted acylated aromatic compounds by converting the acylation reagent with corresponding aromatic compounds in the gas phase is described in general form in JP 07173095.

The use of aryl ketones as the starting material for hair dyes is again described, merely in general form, in DE 19501303.

Nothing is stated about the sensory character of 2,4′-dimethylpropiophenone itself in the documents mentioned.

As already mentioned, 2,4′-dimethylpropiophenone is suitable as a fragrance substance for imparting, modifying and/or reinforcing one, two, three or all the odor notes floral, rosy, rose oxide-like and herbal-like. 2,4′-dimethylpropiophenone is particularly suitable for a corresponding use for imparting, modifying and/or reinforcing both the odor notes rose oxide-like and herbal-like. The fact that this compound has an expressive rose oxide-like and herbal-like odor is particularly surprising as the compound, as, for example, in rose oxide itself, is not a cyclic ether, but an acylated aromatic compound. Generally, a change of the functionalities, even with otherwise structurally similar compounds, leads to clearly different olfactory properties. There is also no structural similarity with typically herbal-like or herbal-similar fragrance substances such as, for example, D-carvon (=(+)-carvon).

rose oxide

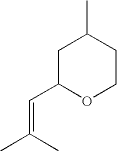

### D-carvon:

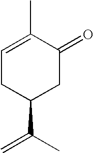

It is also surprising that structurally similar acylated aromatic compounds are described differently in terms of odor. The following list shows selected perfume descriptions, by way of example, of structurally similar acylated aromatic compounds (source: S. Arctander, Perfume and Flavor Chemicals, Vol. I und II, Montclair, N.J., 1969, self-published or K. Bauer, D. Garbe and H. Surburg, Common Fragrance and Flavor Materials, 4th. Ed., Wiley-VCH, Weinheim 2001):
p-methylacetophenone is an important fragrance substance, which has a floral odor of mimosa, cumarin und bitter almond. (S. Arctander, Perfume and Flavor Chemicals, No. 1895, 1969, Montclair, N.J. (USA)).

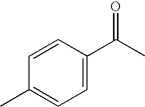

p-methylacetophenone
p-ethylacetophenone is a fragrance substance, which has a sweet, warm, pungent floral odor with a herbal-balsamic undertone. (S. Arctander, Perfume and Flavor Chemicals, No. 1139, 1969, Montclair, N.J. (USA)).

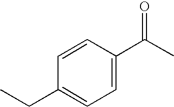

p-ethylacetophenone
p-isopropylacetophenone is a fragrance substance, which has a dry herbaceous and woody odor. In the flavor substance area p-isopropylacetophenone is used in honey and fruit flavors. (S. Arctander, Perfume and Flavor Chemicals, No. 17, 1969, Montclair, N.J. (USA)).

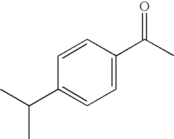

p-isopropylacetophenone
p-isobutylacetophenone is a fragrance substance which is similar to p-methylacetophenone and is distinguished by a woody-hay-like note and high adherence. (S. Arctander, Perfume and Flavor Chemicals, No. 386, 1969, Montclair, N.J. (USA)).

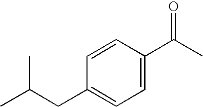

p-isobutylacetophenone

As can be seen in the comparison, none of the acylated aromatic compounds exhibit a rose oxide-like and herbal-like odor note. It was therefore particularly surprising that the acylated aromatic compound to be used according to the invention has a rose oxide-like and herbal-like odor note.

2,4′-dimethylpropiophenone is suitable for the use according to the invention not only because of the described odor notes but also because of its olfactory properties and the material qualities, such as solubility in current cosmetic solvents, compatibility with the current further constituents of products of this type, etc. In addition, the toxicological harmlessness of this fragrance substance underlines the particular suitability of the compound for the use purposes mentioned above and below. Further positive properties of the compound to be used according to the invention and corresponding mixtures comprising this compound are described below in the scope of the uses according to the invention.

Because of the olfactory and material properties mentioned, 2,4′-dimethylpropiophenone is particularly suitable for use as a constituent of a fragrance substance composition of the fougère type or the rose type. Preferably, 2,4′-dimethylpropiophenone is used in these fragrance substance compositions according to a use according to the invention described above.

Rose oxide and herbal-like odor notes are used in diverse perfume compositions, in particular, for example also in fougère or rose perfume themes. The surprisingly (in perfume compositions) floral, rosy, rose oxide-like and herbal-like, in particular rose oxide-like and herbal-like odor profile of 2,4′-dimethylpropiophenone therefore contributes to the fact that this fragrance substance is particularly suitable for corresponding uses in these perfume themes. Above all in conjunction with fragrance substance compositions of the fougère type, a large number of positive properties of 2,4′-dimethylpropiophenone becomes particularly clear. The example described below of a fougère perfume theme (see Example 2) demonstrates in a clear manner the olfactory effect and the positive properties of 2,4′-dimethylpropiophenone. Further advantages of the present invention are therefore found below.

As already mentioned, the fragrance substance 2,4′-dimethylpropiophenone is preferably used according to the invention as a constituent of fragrance substance compositions of the fougère type or the rose type, preferably according to an above-described use. These preferred embodiments are not, however, to restrict the present invention with respect to its aspects. The fragrance substance to be used according to the invention may rather be contained very generally in fragrance substance compositions according to a further aspect of the present invention. Accordingly, the present invention also relates to a fragrance substance composition which contains 2,4′-dimethylpropiophenone and one, two, three or more further fragrance substances, the weight ratio of the total quantity of 2,4′-dimethylpropiophenone to the total quantity of further fragrance substances being in the range of 1:1000 to 1:0.5, preferably in the range of 1:700 to 1:1, particularly preferably in the range of 1:500 to 1:10.

It is particularly preferred in this case if, in a said fragrance substance composition, a quantity of 2,4′-dimethylpropiophenone is contained, which is sufficient to impart, modify and/or reinforce (organoleptically effective quantity) one, two, three or all the odor notes, floral, rosy, rose oxide-like and herbal-like. This preferably applies to a combination of the odor notes rose oxide-like and herbal-like.

As was found in the course of our own investigations, those of the above-described fragrance substance compositions are particularly suitable for the purposes of the present invention which comprise a quantity of 2,4′-dimethylpropiophenone in the range of 0.01 to 65% by weight, preferably from about 0.1 to about 50% by weight, preferably from about 0.5 to about 30% by weight and particularly preferably from about 0.5 to about 25% by weight, based on the total quantity of the fragrance substance composition.

In a mixture with other fragrance substances, the 2,4′-dimethylpropiophenone to be used according to the invention is already able in low doses of the fragrance substance composition to impart an above-described desired odor note.

As already mentioned above it is also preferred if in the fragrance substance compositions according to the invention (mentioned above or described below), a quantity of 2,4′-dimethylpropiophenone is contained, which is sufficient to impart, to modify and/or to reinforce one, two, three or all the odor notes, floral, rosy, rose oxide-like and herbal-like and/or one, two, three or all the after-odor notes floral, rosy, rose oxide-like and herbal-like.

Fragrance substance compositions or perfumed articles are particularly preferred which contain a quantity of 2,4′-dimethylpropiophenone, which is sufficient to impart, to modify and/or to reinforce a rose oxide and herbal-like odor note.

As described, the fragrance substance compositions according to the invention comprise one or more further fragrance substances. According to a further aspect of the present invention, in this case, fragrance substance compositions according to the invention are particularly preferred which comprise at least one further fragrance substance, preferably 2, 3, 4, 5, 6, 7, 8 or more further fragrance substances, wherein the one or the plurality of further fragrance substances are selected from the group consisting of:

alpha-hexylcinnamaldehyde, 2-phenoxyethyl isobutyrate (Phenirat¹), dihydromyrcenol (2,6-dimethyl-7-octen-2-ol), methyl dihydrojasmonate (preferably with a content of cis-isomers of more than 60% by weight) (Hedione⁹, Hedione HC⁹), 4,6,6,7,8,8-hexamethyl-1,3,4,6,7,8-hexahydrocyclopenta[g]benzopyrane (Galaxolid³), tetrahydrolinalool (3,7-dimethyloctan-3-ol), ethyllinalool, benzyl salicylate, 2-methyl-3-(4-tert-butyl-phenyl)propanal (Lilial²), cinnamic alcohol, 4,7-methano-3a,4,5,6,7,7a-hexahydro-5-indenylacetate and/or 4,7-methano-3a,4,5,6,7,7a-hexahydro-6-indenylacetate (Herbaflorat¹), citronellol, vanillin, linalyl acetate, styrolyl acetate (1-phenylethyl acetate), octahydro-2,3,8,8-tetramethyl-2-acetonaphthone and/or 2-acetyl-1,2,3,4,6,7,8-octahydro-2,3,8,8-tetramethylnaphtalene (iso E Super³), hexyl salicylate, 4-tert.-butylcyclohexyl acetate (Oryclone¹), 2-tert.-butylcyclohexylacetate (Agrumex HC¹), alpha-ionone (4-(2,2,6-trimethyl-2-cyclohexen-1-yl)-3-buten-2-one), n-alpha-methylionone, alpha-isomethylionone, coumarin, terpinyl acetate, 2-phenylethyl alcohol, 4-(4-hydroxy-4-methylpentyl)-3-cyclohexencarboxaldehyde (Lyral³), alpha-amylcinnamaldehyde, ethylenbrassylate, (E)- and/or (Z)-3-methylcyclopentadec-5-enone (Muscenon⁹), 15-pentadec-11-enolide and/or 15-pentadec-12-enolide (Globalide¹), 15-cyclopentadecanolide (Macrolide¹), 1-(5,6,7,8-tetrahydro-3,5,5,6,8,8-hexamethyl-2-naphthalenyl)ethanone (Tonalide¹⁰), 2-isobutyl-4-methyltetrahydro-2H-pyran-4-ol (Florol⁹), 2-ethyl-4-(2,2,3-trimethyl-3-cyclopenten-1-yl)-2-buten-1-ol (Sandolen¹), cis-3-hexenylacetate, trans-3-hexenyl acetate, trans-2,cis-6-nonadienol, 2,4-dimethyl-3-cyclohexencarboxaldehyde (Vertocitral¹), 2,4,4,7-tetramethyl-oct-6-en-3-one (Claritone¹), 2,6-dimethyl-5-hepten-1-al (Melonal²), borneol, 3-(3-Isopropylphenyl)butanal (Florhydral²), 2-methyl-3-(3,4-methylendioxyphenyl)propanal (Helional³), 3-(4-ethylphenyl)-2,2-dimethylpropanal (Florazon¹), 7-methyl-2H-1,5-benzodioxepin-3(4H)-one (Calone 19515), 3,3,5-trimethylcyclohexyl acetate (preferably with a content of cis-isomers of 70% by weight or more), and 2,5,5-trimethyl-1,2,3,4,4a,5,6,7-octahydronaphthalene-2-ol (Ambrinol S1). The above-mentioned fragrance substances are accordingly preferably combined in the scope of the present invention with 2,4′-dimethylpropiophenone. Where trade names are given above, these relate to the following sources: ¹Trade name of Symrise GmbH, Germany;²Trade name of Givaudan AG, Switzerland;³Trade name of International Flavors & Fragrances Inc., USA;⁵Trade name of Danisco Seillans S.A., France;⁹Trade name of Firmenich S.A., Switzerland;¹⁰trade name of PFW Aroma Chemicals B.V., Netherlands.

Further fragrance substances, with which the 2,4′-dimethylpropiophenone to be used according to the invention can be advantageously combined, are to be found in S. Arctander, Perfume and Flavor Chemicals, Vol. I und II, Montclair, N.J., 1969, self-published or K. Bauer, D. Garbe and H. Surburg, Common Fragrance and Flavor Materials, 4^{rd}. Ed., Wiley-VCH, Weinheim 2001. Mentioned in detail are:

Extracts from natural raw materials such as essential oils, concretes, absolutes, resins, resinoids, balsams, tinctures such as, for example:
ambra tincture; amyris oil; angelica seed oil; angelica root oil; aniseed oil; valerian oil; basil oil; tree moss absolute; bay oil; mugwort oil; benzoin resin; bergamot oil; beeswax absolute; birch tar oil; bitter almond oil; savory oil; bucco leaf oil; cabreuva oil; cade oil; calamus oil; camphor oil; cananga oil; cardamom oil; cascarilla oil; cassia oil; cassie absolute; castoreum absolute; cedar leaf oil; cedar wood oil; cistus oil; citronella oil; lemon oil; copaiva balsam; copaiva balsam oil; coriander oil; costus root oil; cumin oil; cypress oil; davana oil; dill weed oil; dill seed oil; eau de brouts absolute; oak moss absolute; elemi oil; tarragon oil; eucalyptus-citriodora oil; eucalyptus oil; fenchel oil; spruce needle oil; galbanum oil; galbanum resin; geranium oil; grapefruit oil; guajak wood oil; gurjun balsam; gurjun balsam oil; helichrysum absolute; helichrysum oil; ginger oil; iris root absolute; iris root oil; jasmine absolute; calamus oil; camomile oil blue; camomile oil roman; carrot seed oil; cascarilla oil; pine needle oil; crisped mint oil; caraway oil; labdanum oil; labdanum absolute; labdanum resin; lavandin absolute; lavandin oil; lavender absolute; lavender oil; lemon grass oil; lovage oil; lime oil distilled; lime oil pressed; linaloe oil; litsea cubeba oil; laurel leaf oil; mace oil; marjoram oil; mandarin oil; massoi bark oil; mimosa absolute; musk grain oil; musk tincture; muscatel sage oil; nutmeg oil; myrrh absolute; myrrh oil; myrtle oil; clove leaf oil; clove blossom oil; neroli oil; olibanum absolute; olibanum oil; opopanax oil; orange blossom absolute; orange oil; oregano oil; palmarosa oil; patchouli oil; perilla oil; Peru balsam oil; parsley leaf oil; parsley seed oil; petitgrain oil; peppermint oil; pepper oil; pimento oil; pine oil; poley oil; rose absolute; rosewood oil; rose oil; rosemary oil; sage oil Dalmatian; sage oil Spanish; sandalwood oil; celery seed oil; spike lavender oil; star aniseed oil; styrax oil; marigold oil; fir needle oil; tea tree oil; turpentine oil; thyme oil; tolu balsam; tonka absolute; tuberoses absolute; vanilla extract; violet leaf absolute; verbena oil; vetiver oil; juniper berry oil; wine yeast oil; wormwood oil; wintergreen oil; ylang oil; ysop oil; civet absolute; cinnamon leaf oil; cinnamon bark oil and fractions thereof, or contents isolated therefrom; individual fragrance substances from the group of hydrocarbons such as, for example 3-carene; α-pinene; β-pinene; α-terpinene; γ-terpinene; p-cymol; bisabolene; camphene; caryophyllene; cedrene; farnesene; limonene; longifolene; myrcene; ocimene; valencene; (E,Z)-1,3,5-undecatriene; styrene; diphenylmethane;
aliphatic alcohols such as, for example hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-Pentamethyl-3/4-hepten-2-ol and 3,5,6,6-tetramethyl-4-methyleneheptane-2-ol; (E,Z)-2,6-nonadienol; 3,7-dimethyl-7-methoxyoctane-2-ol; 9-decenol; 10-undecenol; 4-methyl-3-decen-5-ol;
aliphatic aldehydes and acetals thereof such as, for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanaldiethylacetal; 1,1-dimethoxy-2,2,5-trimethyl-4-hexene; citronellyloxyacetaldehyde; 1-(1-methoxy-propoxy)-(E/Z)-3-hexene;
aliphatic ketones and their oximes such as, for example 2-heptanone; 2-octanone; 3-octanone; 2-nonanone; 5-methyl-3-heptanone; 5-methyl-3-heptanonoxime; 2,4,4,7-tetramethyl-6-octen-3-one; 6-methyl-5-hepten-2-one;
aliphatic sulphur-containing compounds such as, for example 3-methylthio-hexanol; 3-methylthiohexylacetate; 3-mercaptohexanol; 3-mercaptohexylacetate; 3-mercaptohexylbutyrate; 3-acetylthiohexylacetate; 1-menthen-8-thiol;
aliphatic nitriles such as, for example 2-nonen acid nitrile; 2-undecanoic acid nitrile; 2-tridecanoic acid nitrile; 3,12-tridecadiene acid nitrile; 3,7-dimethyl-2,6-octadiene acid nitrile; 3,7-dimethyl-6-octenoic acid nitrile;
esters of aliphatic carboxylic acids such as, for example (E)- and (Z)-3-hexenylformate; ethylacetoacetate; isoamyl acetate; hexyl acetate; 3,5,5-trimethylhexylacetate; 3-methyl-2-butenylacetate; (E)-2-hexenylacetate; (E)- and (Z)-3-hexenylacetate; octylacetate; 3-octylacetate; 1-octen-3-ylacetate; ethylbutyrate; butylbutyrate; isoamylbutyrate; hexylbutyrate; (E)- and (Z)-3-hexenyl-isobutyrate; hexylcrotonate; ethylisovalerianate; ethyl-2-methylpentanoate; ethylhexanoate; allylhexanoate; ethylheptanoate; allylheptanoate; ethyloctanoate; ethyl-(E,Z)-2,4-decadienoate; methyl-2-octinate; methyl-2-noninate; allyl-2-isoamyloxyacetate; methyl-3,7-dimethyl-2,6-octadienoate; 4-methyl-2-pentyl-crotonate;
acyclic terpene alcohols such as, for example citronellol; geraniol; nerol; linalool; lavadulol; nerolidol; farnesol; tetrahydrolinalool; tetrahydrogeraniol; 2,6-dimethyl-7-octen-2-ol; 2,6-dimethyloctane-2-ol; 2-methyl-6-methylene-7-octen-2-ol; 2,6-dimethyl-5,7-octadiene-2-ol; 2,6-dimethyl-3,5-octadiene-2-ol; 3,7-dimethyl-4,6-octadiene-3-ol; 3,7-dimethyl-1,5,7-octatriene-3-ol 2,6-dimethyl-2,5,7-octatriene-1-ol; as well as their formates, acetates, propionates, isobutyrates, butyrates, isovalerianates, pentanoates, hexanoates, crotonates, tiglinates and 3-methyl-2-butenoates;
acyclic terpene aldehydes and ketones such as, for example geranial; neral; citronellal; 7-hydroxy-3,7-dimethyloctanal; 7-methoxy-3,7-dimethyloctanal; 2,6,10-trimethyl-9-undecenal; geranylacetone; as well as the dimethyl- and diethylacetals of geranial, neral, 7-hydroxy-3,7-dimethyloctanal;
cyclic terpene alcohols such as, for example menthol; isopulegol; alpha-terpineol; terpineol-4; menthan-8-ol; menthan-1-ol; menthan-7-ol; borneol; isoborneol; linalooloxide; nopol; cedrol; ambrinol; vetiverol; guajol; as well as their formates, acetates, propionates, isobutyrates, butyrates, isovalerianates, pentanoates, hexanoates, crotonates, tiglinates and 3-methyl-2-butenoates;
cyclic terpene aldehydes and ketones such as, for example menthone; isomenthone; 8-mercaptomenthan-3-one; carvone; camphor; fenchone; alpha-lonone; beta-lonone; alpha-n-methylionone; beta-n-methylionone; alpha-isomethylionone; beta-isomethylionone; alpha-iron; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4-a-methanonaphthalen-8(5H)-one; 2-Methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methylcedrylketone);
cyclic alcohols such as, for example 4-tert.-butylcyclohexanol; 3,3,5-trimethyl-cyclohexanol; 3-isocamphylcyclohexanol; 2,6,9-trimethyl-Z2,Z5,E9-cyclododecatriene-1-ol; 2-isobutyl-4-methyltetrahydro-2H-pyran-4-ol;
cycloaliphatic alcohols such as, for example alpha,3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclo-pent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
cyclic and cycloaliphatic ethers such as, for example cineol; cedrylmethylether; cyclododecylmethylether; 1,1-dimethoxycyclododecan; (ethoxymethoxy)cyclododecane; alpha-cedrenepoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methyl propyl)-1,3-dioxane;
cyclic and macrocyclic ketones such as, for example 4-tert.-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert.-pentylcyclohexanone; 5-cyclo-hexadecen-1-one; 6,7-dihydro-1,1,2,3,3-pentamethyl-4(5H)-indanone;
8-cyclohexadecen-1-one 7-cyclohexadecen-1-one; 7/8-cyclohexadecen-1-one; 9-cycloheptadecen-1-one; cyclopentadecanone; cyclohexadecanone;
cycloaliphatic aldehydes such as, for example 2,4-dimethyl-3-cyclohexene carbaldehyde; 2-methyl-4-(2,2,6-trimethyl-cyclohexen-1-yl)-2-butenal; 4-(4-hydroxy-4-methyl pentyl)-3-cyclohexene carbaldehyde; 4-(4-methyl-3-penten-1-yl)-3-cyclo-hexencarbaldehyde;
cycloaliphatic ketones such as, for example 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenylmethyl ketone; methyl-2,6,10-trimethyl-2,5,9-cyclododecatrienyl ketone; tert.-butyl-(2,4-dimethyl-3-cyclohexen-1-yl)ketone;
esters of cyclic alcohols such as, for example 2-tert-butylcyclohexylacetate; 4-tert-butylcyclohexylacetate; 2-tert-pentylcyclohexylacetate; 4-tert-pentylcyclo-hexylacetate; 3,3,5-trimethyl cyclohexyl acetate; decahydro-2-naphthylacetate; 2-cyclopentylcyclopentylcrotonate; 3-pentyltetrahydro-2H-pyran-4-ylacetate; decahydro-2,5,5,8a-tetramethyl-2-naphthylacetate; 4,7-methano-3a,4,5,6,7,7a-hexahydro-5, or 6-indenylacetate; 4,7-methano-3a,4,5,6,7,7a-hexahydro-5, or 6-indenylpropionate; 4,7-methano-3a,4,5,6,7,7a-hexahydro-5, or 6-indenylisobutyrate; 4,7-methanooctahydro-5, or 6-indenylacetate;
esters of cycloaliphatic alcohol such as, for example 1-cyclohexylethylcrotonate;
esters of cycloaliphatic carboxylic acids such as, for example allyl-3-cyclohexylpropionate; allylcyclohexyloxyacetate; cis- and trans-methyl dihydrojasmonate; cis- and trans-methyl jasmonate; methyl-2-hexyl-3-oxocyclopentane carboxylate; ethyl-2-ethyl-6,6-dimethyl-2-cyclohexene carboxylate; ethyl-2,3,6,6-tetramethyl-2-cyclohexene carboxylate; ethyl-2-methyl-1,3-dioxolan-2-acetate;
araliphatic alcohol such as, for example benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
esters of araliphatic alcohols and aliphatic carboxylic acids such as, for example benzyl acetate; benzyl propionate; benzyl isobutyrate; benzyl isovalerianate; 2-phenylethylacetate; 2-phenylethyl propionate; 2-phenylethyl isobutyrate; 2-phenyl-ethyl isovalerianate; 1-phenylethyl acetate; alpha-trichlormethylbenzyl acetate; alpha,alpha-dimethylphenylethyl acetate; alpha,alpha-dimethylphenylethylbutyrate; cinnamyl acetate; 2-phenoxyethylisobutyrate; 4-methoxybenzyl acetate;
araliphatic ethers such as, for example 2-phenylethylmethylether; 2-phenylethylisoamylether; 2-phenylethyl-1-ethoxyethylether; phenylacetaldehyddimethylacetal; phenylacetaldehyde diethyl acetal; hydratropaaldehyde dimethyl acetal; phenylacetaldehyde glycerol acetal; 2,4,6-trimethyl-4-phenyl-1,3-dioxan; 4,4a,5,9b-tetra-hydroindeno[1,2-d]-m-dioxin; 4,4a,5,9b-tetrahydro-2,4-dimethylindeno[1,2-d]-m-dioxin;
aromatic and araliphatic aldehydes such as, for example benzaldehyde; phenyl-acetaldehyde; 3-phenylpropanal; hydratropaaldehyde; 4-methylbenzaldehyde; 4-methyl phenylacetaldehyde; 3-(4-ethylphenyl)-2,2-dimethylpropanal; 2-methyl-3-(4-isopropylphenyl)propanal; 2-methyl-3-(4-tert.-butylphenyl)propanal; 2-methyl-3-(4-isobutylphenyl)propanal; 3-(4-tert.-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
aromatic and araliphatic ketones such as, for example acetophenone; 4-methyl-acetophenone; 4-methoxyacetophenone; 4-tert.-butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3,3,6-hexamethyl-5-indanylmethylketone; 6-tert.-butyl-1,1-dimethyl-4-indanyl-methyl ketone; 1-[2,3-dihydro-1,1,2,6-tetramethyl-3-(1-methylethyl)-1H-5-indenyl]-ethanone; 5′,6′,7′,8′-tetrahydro-3′,5′,5′,6′,8′,8′-hexamethyl-2-acetonaphthone;
aromatic and araliphatic carboxylic acids and esters thereof such as, for example benzoic acid; phenyl acetic acid; methyl benzoate; ethyl benzoate; hexyl benzoate; benzyl benzoate; methylphenyl acetate; ethylphenyl acetate; geranylphenyl acetate; phenylethyl phenylacetate; methyl cinnamate; ethyl cinnamate; benzyl cinnamate; phenylethyl cinnamate; cinnamyl cinnamate; allylphenoxy acetate; methyl salicylate; isoamyl salicylate; hexyl salicylate; cyclohexylsalicylate; cis-3-hexenyl salicylate; benzyl salicylate; phenylethylsalicylate; methyl-2,4-dihydroxy-3,6-dimethyl benzoate; ethyl-3-phenyl glycidate; ethyl-3-methyl-3-phenyl glycidate;
nitrogen-containing aromatic compounds such as, for example 2,4,6-trinitro-1,3-di-methyl-5-tert.-butylbenzene; 3,5-dinitro-2,6-dimethyl-4-tert.-butylacetophenone; cinnamic acid nitrile; 3-methyl-5-phenyl-2-pentanoic acid nitrile; 3-methyl-5-phenyl-pentanoic acid nitrile; methylanthranilate; methyl-N-methylanthranilate; Schiff's bases of methylanthranilate with 7-hydroxy-3,7-dimethyloctanal, 2-methyl-3-(4-tert.-butyl-phenyl)propanal or 2,4-dimethyl-3-cyclohexencarbaldehyde; 6-isopropylchinoline; 6-isobutylchinoline; 6-sec.-butylchinoline; 2-(3-phenylpropyl)pyridine; indol; skatol; 2-methoxy-3-isopropyl pyrazine; 2-isobutyl-3-methoxypyrazine;
phenols, phenyl ethers and phenyl esters such as, for example estragol; anethol; eugenol; eugenylmethyl ether; isoeugenol; isoeugenylmethyl ether; thymol; carvacrol; diphenyl ether; beta-naphthylmethyl ether; beta-naphthylethyl ether; beta-naphthylisobutyl ether; 1,4-dimethoxybenene; eugenylacetate; 2-methoxy-4-methyl-phenol; 2-ethoxy-5-(1-propenyl)phenol; p-kresylphenylacetate;
heterocyclic compounds such as, for example 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
lactones such as, for example 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanol ide; 12-oxa-1,16-hexadecanolide; ethylene-1,12-dodecandioate; ethylene-1,13-tridecandioate; cumarin; 2,3-dihydrocumarin; octahydrocumarin.

As already mentioned several times, 2,4′-dimethylpropiophenone is particularly suitable because of its olfactory properties for use in fragrance substance compositions. The compound can in this case be used combined in corresponding fragrance substance compositions in conjunction with a further individual substance or else a large number of further fragrance substances. The compound may be particularly advantageously combined with other fragrance substances, preferably selected from the fragrance substances already mentioned above, or below, in various different quantity ratios to form novel fragrance substance or perfume compositions

It was already stated above that 2,4′-dimethylpropiophenone is particularly suitable according to the invention for use as a constituent in fragrance substance compositions of the fougère type or the rose type. Accordingly, a further aspect of the present invention relates to a fragrance substance composition (as defined above) with a rose note, which contains at least one further rose fragrance substance, preferably 2, 3, 4 or more further rose fragrance substances, the further rose fragrance substance(s) being selected from the group consisting of:

geranyl formate; geranyl acetate; geranyl propionate; geranyl isobutyrate; geranyl butyrate; geranyl isovalerianate; geranyl tiglinate; geranyl benzoate; citronellyl formate; citronellyl acetate; citronellyl propionate; citronellyl isobutyrate; citronellyl butyrate; citronellyl isovalerianate; citronellyl tiglinate; citronellyl benzoate; phenylethyl alcohol; phenylacetaldehyde; phenylacetaldehyde dimethylacetal; phenylethyl formate; phenylethyl acetate; phenylethyl propionate; phenylethyl isobutyrate; phenylethyl butyrate; phenylethyl pivalate; phenylethyl isovalerianate; phenylethyl-2-ethylbutyrate; phenylethyl tiglinate; phenylethyl benzoate; phenylethyl phenylacetate; 2-phenoxyethyl isobutyrate; geranylmethylether; rose oxide; phenylethylmethylether; phenylethylethylether; phenylethylisoamylether; 2-methoxybenzylethylether; alpha-trichloromethylbenzyl acetate; alpha-3,3-trimethylcyclohexanemethylacetate; 2,4,6-trimethyl-3-cyclohexenmethanol; N,N-diethyl-2-ethyl hexanoic acid amide; 3,7-dimethyloctanol; geranylacetone; linalool; 3,7-dimethyl-1,6-nonadiene-3-ol; nerolidol; farnesol; 9-decenol; 9-decenyl acetate; decanal; 10-undecenal; 10-undecenol; citronellyloxyacetaldehyde; 3,7-dimethyloctanylacetaldehyde; 2-methyl-5-phenylpentanol; 2-methyl-5-phenylpentanal; 3-methyl-5-phenylpentanol; benzophenone; diphenyloxide; diphenylmethane; alpha-damascone; beta-damascone; delta-damascone; gamma-damascone; beta-damascenone; 1-(2,4,4-trimethyl-2-cyclohexenyl)-2-buten-1-one; rose oil; rose absolute; geranium oil, geraniol, nerol, 2-phenylethyl alcohol.

A corresponding use of 2,4′-dimethylpropiophenone is quite particularly preferred in fougère perfume themes, however. A quite particularly preferred embodiment of a fragrance substance composition according to the invention is accordingly a fragrance substance composition with a fougère note, which contains at least one further herbal-herbaceous fragrance substance, preferably 2, 3, 4 or more further herbal-herbaceous fragrance substances, wherein the one or the plurality of further herbal-herbaceous fragrance substances are selected from the group consisting of:

4,7-methano-3a,4,5,6,7,7a-hexahydro-5 (or -6)-indenylacetate; beta-orcin carboxylic acid-methylester; terpinylacetate; terpineol; terpinenol-4; linalool; campher; 1,8-cineol; lavandulol; lavandulylacetate; 1-octen-3-ol; caryophyllene; farnesene; 2-butyl-4,4,6-trimethyl-1,3-dioxan.

As already mentioned, the particularly positive properties of 2,4′-dimethylpropiophenone are described further below in the scope of the uses according to the invention as well as with the aid of the fougère perfume theme example (Example 2) in conjunction with fougère perfume themes or fragrance substance compositions with a fougère note.

Fragrance substance compositions according to the invention or the 2,4′-dimethylpropiophenone to be used according to the invention are preferably used for producing perfumed articles. The sensory properties as well as the substance properties (such as solubility in current solvents and compatibility with current further constituents of products of this type) as well as the toxicological harmlessness of the compound to be used according to the invention underline its particular suitability for said purposes of use. The positive properties described below in conjunction with further uses according to the invention contribute to the fact that the fragrance substance compositions according to the invention are particularly preferably used in washing agents, hygiene or care products, in particular in the area of body and hair care, cosmetics and the household.

Accordingly, a perfumed article is disclosed in the scope of the present invention, which contains a fragrance substance composition according to the invention as defined above. Preferred articles according to the invention are selected from the group consisting of:

Perfume extracts, eau de parfums, eau de toilettes, after-shaves, eau de colognes, pre-shave products, splash colognes, perfumed refresher tissues, perfumes for acid, alkaline and neutral cleaning agents, washing agents, washing tablets, disinfectants, as well as air improvers, aerosols, waxes and polishes, as well as body care agents, bath oils, cosmetic emulsions, such as, for example, skin creams and lotions, sun protection creams and lotions, after sun creams and lotions, hand creams and lotions, foot creams and lotions, depilatory creams and lotions, after-shave creams and lotions, tanning creams and lotions, hair care products, such as, for example, hair sprays, hair gels, strengthening hair lotions, hair rinses, hair dyes, hair shaping agents and hair smoothing agents, hair tonics, hair creams and lotions, deodorants and antiperspirants, products of decorative cosmetics, such as, for example, eye shadows, nail varnishes, make ups, lipsticks, mascara, as well as for candles, lamp oils, joss sticks, insecticides, repellents, propellants.

Fragrance substance compositions according to the invention containing 2,4′-dimethylpropiophenone may however be used in general (for example in concentrated form, in solutions or in modified form described below) for the production of, for example, perfume extracts, eau de parfums, eau de toilettes, after-shaves, eau de cologne, pre-shave products, splash colognes and perfumed refresher tissues and the perfuming of acid, alkaline and neutral cleaning agents, such as, for example, floor cleaners, window glass cleaners, dishwasher detergents, bath and sanitary cleaners, scouring milk, solid and liquid WC cleaners, powdery and foam carpet cleaners. liquid washing agents, powder-like washing agents, pre-treatment washing agents such as bleaching agents, soaking agents and stain removers, laundry softeners, washing soaps, washing tablets, disinfectants, surface disinfection agents and air improvers in liquid, gel-like form or in a form applied on a solid carrier, aerosol sprays, waxes and polishes such as furniture polishes, floor waxes, shoe creams and body care agents, such as, for example, solid and liquid soaps, shower gels, shampoos, shaving soaps, shaving foams, bath oils, cosmetic emulsions of the oil-in-water, of the water-in-oil and of the water-in-oil-in-water type such as, for example, skin creams and lotions, face creams and lotions, sun protection creams and lotions, after sun creams and lotions, hand creams and lotions, foot creams and lotions, depilatory creams and lotions, after-shave creams and lotions, tanning creams and lotions, hair care products, such as, for example, hairsprays, hair gels, strengthening hair lotions, hair rinses, permanent and semi-permanent hair dyes, hair shaping means such as cold waves and hair smoothing agents, hair tonics, hair creams and lotions, deodorants and antiperspirants such as, for example, armpit sprays, roll-ons, deodorant sticks, deodorant creams or products of decorative cosmetics, such as, for example eye shadows, nail varnishes, make ups, lipsticks, mascara as well as for candles, lamp oils, joss sticks, insecticides, repellents, propellants.

That which is stated above applies accordingly to the fragrance substance composition contained in perfumed articles according to the invention and preferred embodiments thereof.

The fragrance substance compositions according to the invention mentioned above or the fragrance substance compositions to be used according to the invention in the corresponding articles may be used in liquid form, undiluted or diluted with a solvent for perfuming. Suitable solvents for this are, for example, ethanol, isopropanol, diethyleneglycolmonoethylether, glycerol, propylene glycol, 1,2-butylene glycol, dipropylene glycol, diethyl phthalate, triethyl citrate, isopropyl myristate etc. For the solvents mentioned the fact applies that these, in the scope of the present text, in the event of the presence of their own olfactory properties, are to be exclusively associated with the constituent “solvent” and not the “fragrance substances”.

The fragrance substance compositions contained in the perfumed articles according to the invention may in this case be absorbed in a preferred embodiment on a carrier substance, which ensures both a fine distribution of the fragrance substances in the product and also a controlled release during the application. Carriers of this type may be porous inorganic materials such as light sulphate, silica gels, zeolites, gypsums, clays, clay granulates, gas concrete etc. or organic materials such as woods and cellulose-based materials.

The fragrance substance compositions according to the invention may also be present micro-encapsulated, spray-dried, as encapsulation complexes or as extrusion products and be added in this form to the product or article to be perfumed.

Optionally, the properties of the fragrance substance compositions modified in this manner can be further optimized by so-called “coating” with suitable materials with regard to a more targeted perfume release, for which purpose wax-like plastics materials, such as, for example, polyvinyl alcohol, are preferably used.

The micro-encapsulation of the fragrance substance compositions may take place, for example, by means of the so-called coacervation method with the aid of capsule materials, for example made of polyurethane-like materials or soft gelatines. The spray-dried perfume oils can be produced, for example, by spray drying of an emulsion or dispersion containing the perfume oil, modified starches, proteins, dextrin and plant rubbers being able to be used as carrier substances. Encapsulation complexes may be produced, for example, by the introduction of dispersions of the fragrance substance composition and cyclodextrins or urea derivatives into a suitable solvent, for example water. Extrusion products may be implemented by means of melting the fragrance substance composition with a suitable wax-like substance and by extrusion with subsequent solidification, optionally in a suitable solvent, for example isopropanol.

The fragrance substance compositions according to the invention can accordingly, as already mentioned, be used in concentrated form, in solutions or in the above-described modified form for the production of the corresponding perfumed articles according to the invention.

Ingredients, with which 2,4′-dimethylpropiophenone can preferably be combined are, for example: preservatives, abrasives, anti-acne agents, agents against skin aging, antibacterial agents, anti-cellulitis agents, antidandruff agents, anti-inflammatories, irritation-preventing agents, irritation-inhibiting agents, antimicrobial agents, antioxidants, astringents, perspiration-inhibiting agents, antiseptic agents, antistatics, binders, buffers, carrier materials, chelating agents, cell stimulants, cleaning agents, care agents, depilatories, surface-active substances, deodorizing agents, antiperspirants, softeners, emulsifiers, enzymes, essential oils, fibres, film formers, fixatives, foaming agents, foam stabilizers, substances for preventing foaming, foam boosters, fungicides, gelling agents, gel-forming means, hair care agents, hair shaping means, hair smoothing means, moisture-dispensing means, moisturizing substances, moisture containing substances, bleaching agents, strengthening agents, stain-removing agents, optically brightening agents, impregnating agents, dirt-repelling agents, friction-reducing agents, lubricants, moisture creams, ointments, opacifiers, plasticising agents, covering means, polishes, brighteners, polymers, powders, proteins, lipid regulating agents, abrasive agents, silicones, skin soothing agents, skin cleaning agents, skin care agents, skin healing agents, skin brightening agents, skin protecting agents, skin softening agents, cooling agents, skin cooling agents, warming agents, skin warming agents, stabilizers, UV-absorbing agents, UV filters, washing agents, fabric softeners, suspended agents, skin tanning agents, thickening agents, vitamins, oils, waxes, fats, phospholipids, saturated fatty acids, singly or multiply unsaturated fatty acids, α-hydroxy acids, polyhydroxy fatty acids, liquifiers, dyes, colour-protecting agents, pigments, anti-corrosives, flavorings, flavor substances, fragrance substances, polyols, surfactants, electrolytes, organic solvents or silicone derivatives.

Further uses according to the invention of 2,4′-dimethylpropiophenone or 2,4′-dimethylpropiophenone-containing mixtures will be described below.

The substantivity of the fragrance substances or of the fragrance substance compositions with regard to the retention thereof on the substrate, in particular hair or textile fibres, is a further important demand, in terms of application technology for surfactant-containing products, on the fragrance substance mixture according to the invention. Surprisingly it has been shown that 2,4′-dimethylpropiophenone is particularly distinguished, in particular for a floral fragrance substance, by a high absorption capacity (inherent adhesion on a substrate) and a high substantivity (capacity for being absorbed from a generally aqueous phase on a substrate or to remain on a substrate even after a washing or rinsing process). This effect is described in further detail below in the framework of an example.

Accordingly, a further aspect of the present invention relates to the use of 2,4′-dimethylpropiophenone as a medium for increasing the substantivity and/or retention of a fragrance substance composition.

Furthermore, 2,4′-dimethylpropiophenone or a mixture containing 2,4′-dimethylpropiophenone can be used as a means for providing (a) hair or (b) textile fibres with one, two, three or all the odor notes floral, rosy, rose oxide-like and herbal-like, preferably with a rose oxide-like and herbal-like odor note.

The 2,4′-dimethylpropiophenone to be used according to the invention is therefore particularly suitable for use in surfactant-containing products.

It is preferred if the product is one of the following:

an acid, alkaline or neutral cleaning agent, selected in particular from the group consisting of all-purpose cleaners, floor cleaners, window glass cleaners, dishwasher detergents, bath and sanitary cleaners, scouring milk, solid and liquid WC cleaners, powdery and foam carpet cleaners. liquid washing agents, powder-like washing agents, pre-treatment washing agents such as bleaching agents, soaking agents and stain removers, laundry softeners, washing soaps, washing tablets, disinfectants, surface disinfection agents,

an air improver in liquid, gel-like form or in a form applied on a solid carrier, or as an aerosol spray,

a wax or a polish, which is in particular selected from the group consisting of furniture polishes, floor waxes and shoe creams, or

a body care agent, which is selected in particular from the group consisting of shower gels and shampoos.

According to a further aspect of the present invention, the use of 2,4′-dimethylpropiophenone is disclosed as a means for increasing the complex stability of a fragrance substance composition.

It was surprisingly shown in our own investigations that 2,4′-dimethylpropiophenone, in the fragrance substance compositions according to the invention, preferably in one of the configurations disclosed as preferred above,—expressed figuratively—brings about a “moving together” of the individual constituents of the composition, in other words, improves and extends the complex stability. This means that, for example, the evaporation behavior of the individual fragrance substances or the fragrance substance components is linearized and synchronized in an olfactory respect. This effect is shown above all in the preferred fragrance substance compositions of the fougère type.

In addition, a use of 2,4′-dimethylpropiophenone is disclosed as a means for changing, masking and/or reducing a medical odor note in a fragrance substance composition, preferably in a fragrance substance composition according to the invention. In our own investigations it was again shown using the example of a fragrance substance composition of the fougère type that 2,4′-dimethylpropiophenone takes the medical perfume character from the fragrance substance composition or the herbal notes contained therein, or substantially reduces it. As a result, 2,4′-dimethylpropiophenone promotes the balance of the fragrance substance composition and produces a riper effect in terms of perfume. A use according to the invention in a fragrance substance composition of the fougère type is therefore particularly preferred.

Fragrance substance compositions with a rose oxide and herbal-like head note are frequently looked for, and these should simultaneously have a distinct blooming (odor from an aqueous solution). As is shown below in the scope of the embodiments, the 2,4′-dimethylpropiophenone to be used according to the invention is also suitable for this use purpose.

A further aspect of the present invention accordingly relates to the use of 2,4′-dimethylpropiophenone as a means for increasing the odor of other fragrance substances perceived over a surfactant-containing aqueous solution.

The 2,4′-dimethylpropiophenone to be used according to the invention as a fragrance substance can be produced by a Friedel-Craft acylation described in current standard works. In a preferred production method, toluene is used to excess and is therefore used both as a reagent and as a solvent. The preferred Lewis acid aluminum chloride is preferably provided and the reaction temperature is controlled by the dosing speed of the isobutyric acid halogenide, preferably the isobutyric acid chloride. The regioisomeric meta-(1.7%) and ortho-(3.8%) compounds are produced as by-products.

The reaction can be shown by way of example using the following plan:

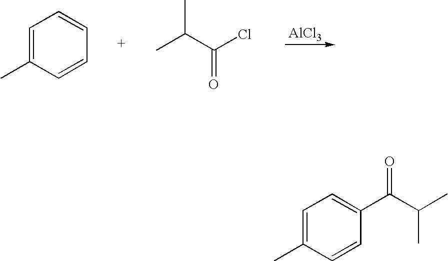

This means that, depending on acylation conditions, the reaction product may also contain the isomeric ortho- and meta-ketones. Accordingly, a fragrance substance composition according to the invention or an article perfumed according to the invention may also contain the isomeric ortho- and meta-ketones.

A fragrance substance composition according to the invention is produced according to the invention in that 2,4′-dimethylpropiophenone is mixed with the further fragrance substance(s) and optionally further constituents of the fragrance substance composition.

According to a preferred embodiment, a fragrance substance composition according to the invention is produced as described above, but the 2,4′-dimethylpropiophenone is used in a quantity which is sufficient to impart, to modify and/or to reinforce in the fragrance substance composition one, two, three or all of the odor notes floral, rosy, rose oxide-like and herbal-like, preferably both of the odor notes rose oxide-like and herbal-like.

That which is stated above applies accordingly to the further constituents or fragrance substances to be preferably selected in addition to 2,4′-dimethylpropiophenone.

As already mentioned, 2,4′-dimethylpropiophenone may preferably be used according to the invention for use as a means for providing hair or textile fibres with a correspondingly desired odor note.

Accordingly, a further aspect of the present invention relates to a method for providing (a) hair or (b) textile fibres with one, two, three or all the odor notes floral, rosy, rose oxide-like and herbal-like, preferably with a rose oxide-like and herbal-like odor note, comprising the following steps:

i) providing a mixture, comprising 2,4′-dimethylpropiophenone or a fragrance substance composition according to the invention as defined above,
ii) applying the mixture to (a) hair or (b) the textile fibres.

The invention will be described below in more detail with the aid of examples.

Unless stated otherwise, all data, in particular percentages, relate to the weight. Abbreviations used: krist.=crystallized; nat.=natural

### EXAMPLE 1

### Preparation of 2,4′-dimethylpropiophenone

368 g toluene (4 mol) and 200 g (1.50 mol) AlCl₃ are provided at 0° C. 159 g (1.49 mol) isobutyric acid chloride are then added stepwise at 0° C. within 2 hours. At the end of the dosing, stirring takes place for 30 minutes at a temperature of 0° C. to 8° C. The reaction batch is placed on 318 g water/440 g ice. The aqueous phase is separated off, the organic phase is rewashed twice in each case with 160 g water. 458 g 2,4′-dimethylpropiophenone are obtained crude in toluene and are adjusted with about 1 g saturated sodium bicarbonate solution to a pH of 8.5. After toluene and water have been distilled off, the product distils over at a head temperature of 85 to 88° C. and a vacuum of 2 mbar. 225 g of 2,4′-dimethylpropiophenone distillate are obtained.

The isomeric distribution in the quenched reaction batch, according to the GC evaluation (capillary column DB-1, Kaltin injection) is as follows:

2,4′-dimethylpropiophenone (para) 94.4%, 2,3′-dimethylpropiophenone (meta) 1.7% (odor: weaker than para and ortho, rosy, technical-grade) and 2,2′-dimethylpropiophenone (ortho) 3.8% (odor: stronger than meta, rosy, powdery, dusty).

The 2,4′-dimethylpropiophenone yield based on isobutyric acid chloride is 87.9% of the theoretical yield.

Spectroscopic and Spectrometric Data of 2,4′-dimethylpropiophenone:

¹H-NMR (CDCl₃, 400 MHz, TMS=0 ppm): δ=1.21 ppm (d, 2-CH₃, 6H), 2.41 (s, —CH₃, 3H), 3.54 (sept, —CH, 1H), 7.26 (m, 2—CH, 2H), 7.86 (m, 2—CH, 2H).

¹³C-NMR (CDCl₃, 75 MHz): δ=19.22 ppm (2 C), 21.56, 35.20, 128.46 (2 C), 129.29 (2 C), 133.70, 143.51, 204.16.

MS (m/e, %): 162 (M, 8%), 120 (10), 119 (100), 91 (33), 65 (12), 39 (5).

### EXAMPLE 2

### Fragrance Substance Composition with and without 2,4′-dimethylpropiophenone

| | Perfume oil AE | Perfume oil AB |
|---|---|---|
| n-dodecanal | 3 | 3 |
| 2-methylundecanal | 7 | 7 |
| Vertocitral¹⁾ | 10 | 10 |
| galbanum resin | 3 | 3 |
| styrolylacetate | 7 | 7 |
| terpinylacetate | 30 | 30 |
| citral | 10 | 10 |
| orange oil, Italian | 20 | 20 |
| Claritone ®¹⁾ | 10 | 10 |
| petitgrain oil | 20 | 20 |
| lavandin oil Grosso | 100 | 100 |
| spike oil, Spanish | 100 | 100 |
| rosmary oil | 60 | 60 |
| sage oil, Spanish | 30 | 30 |
| dill weed oil | 10 | 10 |
| mugwort oil | 10 | 10 |
| Lyral ®²⁾ | 50 | 50 |
| Hedione³⁾ | 50 | 50 |
| methyloctincarbonate | 1 | 1 |
| Herbaflorat¹⁾ | 25 | 25 |
| herbylpropionate | 20 | 20 |
| Iso E Super²⁾ | 70 | 70 |
| vetiver oil Java | 10 | 10 |
| Vetikolacetat ®¹⁾ | 5 | 5 |
| patchouli oil | 30 | 30 |
| Evernyl⁴⁾ | 5 | 5 |
| Ambroxid krist.¹⁾ | 4 | 4 |
| 2,4′-dimethyl-propiophenone | 70 | — |
| DPG (dipropylene glycol) | 230 | 300 |
| | 1000 | 1000 |

¹⁾Trade name of the company Symrise, Holzminden, Germany
²⁾Trade name of the company IFF, New Jersey, USA
³⁾Trade name of the company Firmenich, Geneva, Switzerland
⁴⁾Trade name of the company Givaudan, Zürich, Switzerland

The demonstration examples of the perfume oils AE/AB are fragrance substance compositions, especially perfume compositions of the fougère type, consisting of natural, synthetic-identical to natural and fully synthetic fragrance substances.

The profile of the perfume is selected in such a way that substances with a leaf green odor, components smelling of citrus fruits, herby-herbaceous-spicy and medically smelling herb oils and materials with dry-woody echoes come together in harmony in terms of perfume.

The perfume impression does not exhibit the desired complexity in all the phases of the evaporation owing to different chemical-physical properties of the individual components.

As already mentioned in the scope of the description of the present invention, the use of 2,4′-dimethylpropiophenone as a fragrance substance offers numerous advantages. 2,4′-dimethylpropiophenone also brings about (in the figurative sense) a “moving together” of the individual constituents in the selected concentration of the embodiment. This means that the evaporation behavior of the individual components is linearized and synchronized in olfactory terms. The complex stability of the fragrance substance composition is extended thereby.

The herbal note of the odor profile of 2,4′-dimethylpropiophenone is distinguished more by a spicy, green character in the direction of herbaceous and is less to be regarded as medical. In the fragrance substance composition, the spicy and green, herbaceous aspects are reinforced by 2,4′-dimethylpropiophenone and the medical aspects are reduced. 2,4′-dimethylpropiophenone therefore acts in a harmonizing manner on the herbal notes and removes from them or reduces the medical perfume character. 2,4′-dimethylpropiophenone promotes the balance of the fragrance substance composition and produces a riper effect in terms of perfume.

The further application technology properties, which are better in comparison to previous market products, of 2,4′-dimethylpropiophenone will be described below in some examples.

### EXAMPLE 3

### Stability and Recovery Rates of 2,4′-dimethylpropiophenone and D-carvon

The stability and recovery rates are determined by storage of the fragrance substances to be compared (2,4′-dimethylpropiophenone and D-carvone, nat.) in the corresponding matrix over a specific timeframe.

TABLE 1
Recovery rates (storage at 38° C., details in %)

| | 2,4′-dimethylpropiophenone | | D-carvone, nat. | |
|---|---|---|---|---|
| | 1 Month | 2 Months | 1 Month | 2 Months |
| EDT (Eau-de- | 100 | 100 | 100 | 99 |
| toilette) | | | | |
| Antiperspirant | 98 | 94 | 89 | 86 |
| Soap | 100 | 100 | 100 | 98 |
| Shampoo | 96 | 96 | 92 | 87 |
| Washing powder | 99 | 91 | 75 | 67 |
| Hair coloration | 100 | 99 | 91 | 78 |
| Bleach | 16 | 15 | 0 | 0 |

The recovery rates of 2,4′-dimethylpropiophenone are clearly better than those of D-carvone.

The formulations of the aforementioned matrices are as follows:

### EDT (Eau-de-Toilette):

| | |
|---|---|
| ethanol | 96% |
| fragrance substance | 4% |

### Antipersipirant:

| | |
|---|---|
| demineralized water | 59.4% |
| hydroxyethylcellulose | 0.3% |
| aluminum zirconium pentachlorohydrate | 37.5% |
| C12-15 Pareth-12 | 1.2% |
| PEG-40 hydrogenated castor oil and propylene glycol | 0.8% |
| fragrance substance | 0.8% |

### Shampoo:

| | |
|---|---|
| sodium lauryl ether sulphate | 20% |
| (for example Texapon NSO, company Cognis | |
| Deutschland GmbH) | |
| sodium chloride | 1.4% |
| citric acid | 1.3% |
| phenoxyethanol, methyl-, ethyl-, butyl-, | 0.5% |
| and propylparaben | |
| water | 76.3% |
| fragrance substance | 0.5% |

Washing Powder:

| | |
|---|---|
| linear Na-alkyl benzene sulfonate | 8.7% |
| ethoxylated fatty alcohol C12-18 (7 EO) | 4.6% |
| Na soap | 3.2% |
| defoamer | 3.8% |
| DOW CORNING(R) 2-4248S | |
| POWDERED ANTIFOAM, | |
| silicone oil on zeolite as the carrier material | |
| zeolite 4A | 28.3% |
| Na carbonate | 11.5% |
| Na salt of a copolymer of acrylic and maleic acid (Sokalan CP5) | 2.4% |
| Na silicate | 3.0% |
| carboxymethylcellulose | 1.2% |
| Dequest 2066 | 2.8% |
| ([[(phosphonomethyl)imino]bis[(ethylenenitrilo)bis | |
| (methylene)]]tetrakis-phosphonic acid, sodium salt) | |
| optical brighteners | 0.2% |
| Na sulphate | 6.4% |
| protease | 0.4% |
| sodium perborate tetrahydrate | 22.0% |
| TAED | 1.0% |
| fragrance substance | 0.5% |

### Hair Coloration/Hair Dye:

| | |
|---|---|
| cetyl alcohol, oleyl alcohol, cetearyl alcohol and stearic acid | 21.0% |
| Ceteareth-25 | 4.0% |
| Laureth-8 | 10.0% |
| sodium cetearyl sulfate | 1.0% |
| demineralized water | 49.0% |
| Polyquaternium-6 | 4.0% |
| fragrance substance | 0.8% |
| ammonia solution (20%) | 10.2% |

### Bleach:

| | |
|---|---|
| demineralized water | 47.60% |
| NaOH | 0.5% |
| Dowfax 3B3 (about 45%) | 0.8% |
| lauryl ether carboxylic acid | 0.8% |
| stabilizer (phosphonates) | 0.2% |
| Na hypochlorite solution (content about 10.3%) | 50.0% |
| fragrance substance | 0.1% |

The percentage details given here in each case relate to, if not otherwise mentioned, the total weight of the perfumed article or the fragrance substance composition.

### EXAMPLE 4

### Sensory data of 2,4′-dimethylpropiophenone in comparison to D-carvone

TABLE 2
Sensory data for 2,4′-dimethylpropiophenone
in comparison to D-carvone

| | | 2,4′- | D-carvone, |
|---|---|---|---|
| Test | Scale | dimethylpropiophenone | nat. |
| Odor threshold value | 1-13 | 4 | 4 |
| (from diethyl phthalate) | | | |
| Odor threshold value | 5-13 | 7.1 | 6.9 |
| (from air) | | | |
| Impact (from the | 1-9 | 5 | 5 |
| fragrance strip) | | | |
| Intensity (on air) | 1-9 | 6 | 6 |

The evaluation took place in each case by means of a sensory panel with the aid of the given scales, 13 or 9 being the best possible evaluations.

The odor threshold value (from diethyl phthalate and from air) and the impact (from the fragrance strip) and the intensity (on air) of the compared fragrance substances are to be assessed as virtually the same.

### EXAMPLE 5

### Substantivity/retention of 2,4′-dimethylpropiophenone

As already mentioned, for use in surfactant-containing products, the substantivity relative to the substrate or the retention on the substrate are important application technology demands on the fragrance substance composition, in particular in the case of hair or textile fibres as the substrate. 2,4′-dimethylpropiophenone is distinguished, in particular for a floral fragrance substance, by a high absorption capacity and a high substantivity. This effect is shown in particular on substrates such as skin, hair and textile fibres (for example wool, cotton, linen, synthetic fibres).

A performance test was carried out: the pH of the shampoo basic compound (description see above) was about 6. From this 100 ml of a 20% by weight aqueous shampoo solution was produced. Two hair strands were washed together for 2 minutes in this shampoo solution and then rinsed for 20 seconds under flowing hand-hot water. One hair strand was packed wet in aluminum foil and the second hair strand was dried with a hair drier. The two hair strands were assessed in terms of odor by a panel on a scale from 0-6, 6 being the best possible evaluation.

TABLE 3
Shampoo

| | 2,4′-dimethylpropiophenone | D-carvone, nat. |
|---|---|---|
| Damp hair | 2.6 | 2.3 |
| Dry hair | 1.9 | 0.9 |

2,4′-dimethylpropiophenone therefore proved to be superior.

### EXAMPLE 6

### Blooming properties of 2,4′-dimethylpropiophenone

Fragrance substance mixtures are frequently looked for with a rose oxide-like and herbal-like head note, and these—as mentioned above—should simultaneously have a distinct blooming (odor from an aqueous solution). In this regard, 2,4′-dimethylpropiophenone was therefore also compared in a direct performance test with D-carvone, nat. (panel evaluation 1-9, 9 being the best possible evaluation):

| | | 2,4′-dimethylpropiophenone | D-carvone, nat. |
|---|---|---|---|
| Blooming | from water | 7 | 7 |

The compound therefore also accordingly proved to be a substance with a strong “blooming”.

Because of the positive properties shown of 2,4′-dimethylpropiophenone, articles particular preferably to be perfumed are therefore washing agents, hygiene or care products, in particular in the area of body care, cosmetics and the household.

### SPECIFIC EMBODIMENTS

Specific embodiment one comprises a use of 2,4′-dimethylpropiophenone as a fragrance substance.

Specific embodiment two comprises the use as in specific embodiment one for imparting, modifying and/or reinforcing one, two, three or all the odor notes floral, rosy, rose oxide-like and herbal-like, preferably both of the odor notes rose oxide-like and herbal-like.

Specific embodiment three comprises the use of 2,4′-dimethylpropiophenone, preferably as in any one of the preceding specific embodiments, as a component of a fragrance substance composition of the fougère type or the rose type.

Specific embodiment four comprises a fragrance substance composition containing 2,4′-dimethylpropiophenone and one, two, three or more further fragrance substances, wherein the weight ratio of the total quantity of 2,4′-dimethylpropiophenone to the total quantity of further fragrance substances is in the range of 1:1000 to 1:0.5, preferably in the range of 1:700 to 1:1, particularly preferably in the range of 1:500 to 1:10.

Specific embodiment five comprises the fragrance substance composition as in specific embodiment four, wherein the quantity of 2,4′-dimethylpropiophenone is sufficient to impart, to modify and/or to reinforce one, two, three or all the odor notes floral, rosy, rose oxide-like and herbal-like, preferably both of the odor notes rose oxide-like and herbal-like.

Specific embodiment six comprises the fragrance substance composition as in either of specific embodiments four and five, wherein the fragrance substance composition comprises a quantity of 2,4′-dimethylpropiophenone in the range of 0.01 to 65% by weight, preferably from about 0.1 to about 50% by weight, preferably from about 0.5 to about 30% by weight and particularly preferably from about 0.5 to about 25% by weight, based on the total quantity of the fragrance substance composition.

Specific embodiment seven comprises the fragrance substance composition as in any one of specific embodiments four, five, or six, comprising at least one further fragrance substance, preferably 2, 3, 4, 5, 6, 7, 8 or more further fragrance substances, wherein the further fragrance substance(s) are selected from the group consisting of:

alpha-hexylcinnamaldehyde, 2-phenoxyethyl isobutyrate, dihydromyrcenol, methyl dihydrojasmonate, 4,6,6,7,8,8-hexamethyl-1,3,4,6,7,8-hexahydrocyclopenta[g]benzopyrane, tetrahydrolinalool, ethyllinalool, benzylsalicylate, 2-methyl-3-(4-tert-butyl-phenyl)propanal, cinnamic alcohol, 4,7-methano-3a,4,5,6,7,7a-hexahydro-5-indenylacetate and/or 4,7-methano-3a,4,5,6,7,7a-hexahydro-6-indenylacetate, citronellol, vanillin, linalylacetate, styrolylacetate, octahydro-2,3,8,8-tetramethyl-2-acetonaphthone and/or 2-acetyl-1,2,3,4,6,7,8-octahydro-2,3,8,8-tetramethylnaphthalene, hexylsalicylate, 4-tert.-butylcyclohexylacetate, 2-tert.-butylcyclohexylacetate, alpha-ionone, n-alpha-methylionone, alpha-isomethylionone, coumarin, terpinyl acetate, 2-phenylethyl alcohol, 4-(4-hydroxy-4-methyl pentyl)-3-cyclohexencarboxaldehyde, alpha-amylcinnamaldehyde, ethylenebrassylate, (E)- and/or (Z)-3-methylcyclopentadec-5-enone, 15-pentadec-11-enolide and/or 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H-pyran-4-ol, 2-ethyl-4-(2,2,3-trimethyl-3-cyclopenten-1-yl)-2-buten-1-ol, cis-3-hexenyl acetate, trans-3-hexenyl acetate, trans-2,cis-6-nonadienol, 2,4-dimethyl-3-cyclohexenecarboxaldehyde, 2,4,4,7-tetramethyl-oct-6-en-3-one, 2,6-dimethyl-5-hepten-1-al, borneol, 3-(3-Isopropylphenyl)butanal, 2-methyl-3-(3,4-methylendioxyphenyl)propanal, 3-(4-ethylphenyl)-2,2-dimethylpropanal, 7-methyl-2H-1,5-benzodioxepin-3(4H)-one, 3,3,5-trimethylcyclohexyl acetate and 2,5,5-trimethyl-1,2,3,4,4a,5,6,7-octahydronaphthalene-2-ol.

Specific embodiment eight comprises the fragrance substance composition as in any one of specific embodiments four, five, six, or seven with a rose note, comprising at least one further rose fragrance substance, preferably 2, 3, 4 or more further rose fragrance substances, wherein the or the further rose fragrance substances are selected from the group consisting of:

geranyl formate; geranyl acetate; geranyl propionate; geranyl isobutyrate; geranyl butyrate; geranyl isovalerianate; geranyl tiglinate; geranyl benzoate; citronellyl formate; citronellyl acetate; citronellyl propionate; citronellyl isobutyrate; citronellyl butyrate; citronellyl isovalerianate; citronellyl tiglinate; citronellyl benzoate; phenylethyl alcohol; phenylacetaldehyde; phenylacetaldehyde dimethylacetal; phenylethyl formate; phenylethyl acetate; phenylethyl propionate; phenylethyl isobutyrate; phenylethyl butyrate; phenylethyl pivalate; phenylethyl isovalerianate; phenylethyl-2-ethylbutyrate; phenylethyl tiglinate; phenylethyl benzoate; phenylethyl phenylacetate; 2-phenoxyethyl isobutyrate; geranylmethylether; rose oxide; phenylethyl methylether; phenylethyl ethylether; phenylethyl isoamylether; 2-methoxybenzylethylether; alpha-trichlormethylbenzylacetate; alpha-3,3-trimethylcyclohexanemethylacetate; 2,4,6-trimethyl-3-cyclohexenmethanol; N,N-diethyl-2-ethyl hexanoic acid amide; 3,7-dimethyloctanol; geranylacetone; linalool; 3,7-dimethyl-1,6-nonadiene-3-ol; nerolidol; farnesol; 9-decenol; 9-decenyl acetate; decanal; 10-undecenal; 10-undecenol; citronellyloxyacetaldehyde; 3,7-dimethyloctanylacetaldehyde; 2-methyl-5-phenylpentanol; 2-methyl-5-phenylpentanal; 3-methyl-5-phenylpentanol; benzophenone; diphenyloxide; diphenylmethane; alpha-damascone; beta-damascone; delta-damascone; gamma-damascone; beta-damascenone; 1-(2,4,4-trimethyl-2-cyclohexenyl)-2-buten-1-one; rose oil; rose absolute; geranium oil, geraniol, nerol, 2-phenylethyl alcohol.

Specific embodiment nine comprises the fragrance substance composition as in any one of specific embodiments four, five, six, or seven with a fougère note, comprising at least one further herbal-herbaceous fragrance substance, preferably 2, 3, 4 or more further herbal-herbaceous fragrance substances, wherein the or the further herbal-herbaceous fragrance substances are selected from the group consisting of:

4,7-methano-3a,4,5,6,7,7a-hexahydro-5 (or -6)-indenylacetates; beta-orcin carboxylic acid-methylester; terpinyl acetate; terpineol; terpinenol-4; linalool; campher; 1,8-cineol; lavandulol; lavandulyl acetate; 1-octen-3-ol; caryophyllene; farnesene; 2-butyl-4,4,6-trimethyl-1,3-dioxan.

Specific embodiment ten comprises a perfumed article, comprising a fragrance substance composition as in any one of specific embodiments four to nine, wherein the article is selected from the group consisting of:

Perfume extracts, eau de parfums, eau de toilettes, after-shaves, eau de colognes, pre-shave products, splash colognes, perfumed refresher tissues, perfumes for acid, alkaline and neutral cleaning agents, washing agents, washing tablets, disinfectants as well as air improvers, aerosol sprays, waxes and polishes, as well as body care agents, bath oils, cosmetic emulsions, hair care products, deodorants and antiperspirants, products of decorative cosmetics as well as of candles, lamp oils, joss sticks, insecticides, repellents, propellants.

Specific embodiment eleven comprises the use of 2,4′-dimethylpropiophenone or a mixture containing 2,4′-dimethylpropiophenone as a means for providing (a) hair or (b) textile fibres with one, two, three or all of the odor notes floral, rosy, rose oxide-like and herbal-like, preferably with a rose oxide-like and herbal-like odor note.

Specific embodiment twelve comprises the use of 2,4′-dimethylpropiophenone as a means for increasing the substantivity and/or retention of a fragrance substance composition.

Specific embodiment thirteen comprises a method for producing a fragrance substance composition as in any one of specific embodiments four to nine, comprising the following step:

mixing 2,4′-dimethylpropiophenone with the or the further fragrance substances and optionally with further constituents of the fragrance substance composition.

Specific embodiment fourteen comprises the method as in specific embodiment thirteen, wherein the 2,4′-dimethylpropiophenone is used in a quantity which is sufficient in the fragrance substance composition to impart, to modify and/or to reinforce one, two, three or all the odor notes floral, rosy, rose oxide-like and herbal-like, preferably both of the odor notes rose oxide-like and herbal-like.

We claim:

1. A method of fragrancing or enhancing fragrance of matter comprising adding 2,4′-dimethylpropiophenone as a fragrance substance.

2. The method as claimed in claim 1 further comprising imparting, modifying and/or reinforcing one, two, three or all the odor notes floral, rosy, rose oxide-like and herbal-like.

3. The method as claimed in claim 1, wherein the 2,4′-dimethylpropiophenone is a component of a fragrance substance composition of the fougère type or the rose type.

4. A fragrance substance composition containing 2,4′-dimethylpropiophenone and one, two, three or more further fragrance substances, wherein the weight ratio of the total quantity of 2,4′-dimethylpropiophenone to the total quantity of further fragrance substances is in the range of 1:1000 to 1:0.5.

5. The fragrance substance composition as claimed in claim 4, wherein the quantity of 2,4′-dimethylpropiophenone is sufficient to impart, to modify and/or to reinforce one, two, three or all the odor notes floral, rosy, rose oxide-like and herbal-like.

6. The fragrance substance composition as claimed in claim 4, wherein the fragrance substance composition comprises a quantity of 2,4′-dimethylpropiophenone in the range of 0.01 to 65% by weight, based on the total quantity of the fragrance substance composition.

7. The fragrance substance composition as claimed in claim 4, comprising at least one further fragrance substance, wherein the further fragrance substance(s) are selected from the group consisting of:
alpha-hexylcinnamaldehyde, 2-phenoxyethyl isobutyrate, dihydromyrcenol, methyl dihydrojasmonate, 4,6,6,7,8,8-hexamethyl-1,3,4,6,7,8-hexahydrocyclopenta[g]benzopyrane, tetrahydrolinalool, ethyllinalool, benzylsalicylate, 2-methyl-3-(4-tert-butyl-phenyl)propanal, cinnamic alcohol, 4,7-methano-3a,4,5,6,7,7a-hexahydro-5-indenylacetate and/or 4,7-methano-3a,4,5,6,7,7a-hexahydro-6-indenylacetate, citronellol, vanillin, linalylacetate, styrolylacetate, octahydro-2,3,8,8-tetramethyl-2-acetonaphthone and/or 2-acetyl-1,2,3,4,6,7,8-octahydro-2,3,8,8-tetramethylnaphthalene, hexylsalicylate, 4-tert.-butylcyclohexylacetate, 2-tert.-butylcyclohexylacetate, alpha-ionone, n-alpha-methylionone, alpha-isomethylionone, coumarin, terpinyl acetate, 2-phenylethyl alcohol, 4-(4-hydroxy-4-methylpentyl)-3-cyclohexencarboxaldehyde, alpha-amylcinnamaldehyde, ethylenebrassylate, (E)- and/or (Z)-3-methylcyclopentadec-5-enone, 15-pentadec-11-enolide and/or 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H-pyran-4-ol, 2-ethyl-4-(2,2,3-trimethyl-3-cyclopenten-1-yl)-2-buten-1-ol, cis-3-hexenyl acetate, trans-3-hexenyl acetate, trans-2,cis-6-nonadienol, 2,4-dimethyl-3-cyclohexenecarboxaldehyde, 2,4,4,7-tetramethyl-oct-6-en-3-one, 2,6-dimethyl-5-hepten-1-al, borneol, 3-(3-Isopropylphenyl)butanal, 2-methyl-3-(3,4-methylendioxyphenyl)propanal, 3-(4-ethylphenyl)-2,2-dimethylpropanal, 7-methyl-2H-1,5-benzodioxepin-3(4H)-one, 3,3,5-trimethylcyclohexyl acetate and 2,5,5-trimethyl-1,2,3,4,4a,5,6,7-octahydronaphthalene-2-ol.

8. The fragrance substance composition as claimed in claim 5, wherein the composition has a rose note, comprising at least one further rose fragrance substance, wherein the or the further rose fragrance substances are selected from the group consisting of:
geranyl formate; geranyl acetate; geranyl propionate; geranyl isobutyrate; geranyl butyrate; geranyl isovalerianate; geranyl tiglinate; geranyl benzoate; citronellyl formate; citronellyl acetate; citronellyl propionate; citronellyl isobutyrate; citronellyl butyrate; citronellyl isovalerianate; citronellyl tiglinate; citronellyl benzoate; phenylethyl alcohol; phenylacetaldehyde; phenylacetaldehyde dimethylacetal; phenylethyl formate; phenylethyl acetate; phenylethyl propionate; phenylethyl isobutyrate; phenylethyl butyrate; phenylethyl pivalate; phenylethyl isovalerianate; phenylethyl-2-ethylbutyrate; phenylethyl tiglinate; phenylethyl benzoate; phenylethyl phenylacetate; 2-phenoxyethyl isobutyrate; geranylmethylether; rose oxide; phenylethyl methylether; phenylethyl ethylether; phenylethyl isoamylether; 2-methoxybenzylethylether; alpha-trichlormethylbenzylacetate; alpha-3,3-trimethylcyclohexanemethylacetate; 2,4,6-trimethyl-3-cyclohexenmethanol; N,N-diethyl-2-ethyl hexanoic acid amide; 3,7-dimethyloctanol; geranylacetone; linalool; 3,7-dimethyl-1,6-nonadiene-3-ol; nerolidol; farnesol; 9-decenol; 9-decenyl acetate; decanal; 10-undecenal; 10-undecenol; citronellyloxyacetaldehyde; 3,7-dimethyloctanylacetaldehyde; 2-methyl-5-phenylpentanol; 2-methyl-5-phenylpentanal; 3-methyl-5-phenylpentanol; benzophenone; diphenyloxide; diphenylmethane; alpha-damascone; beta-damascone; delta-damascone; gamma-damascone; beta-damascenone; 1-(2,4,4-trimethyl-2-cyclohexenyl)-2-buten-1-one; rose oil; rose absolute; geranium oil, geraniol, nerol, and 2-phenylethyl alcohol.

9. The fragrance substance composition as claimed in claim 4, wherein the composition has a fougère note, comprising at least one further herbal-herbaceous fragrance substance, preferably 2, 3, 4 or more further herbal-herbaceous fragrance substances, wherein the or the further herbal-herbaceous fragrance substances are selected from the group consisting of:
4,7-methano-3a,4,5,6,7,7a-hexahydro-5 (or -6)-indenylacetates; beta-orcin carboxylic acid-methylester; terpinyl acetate; terpineol; terpinenol-4; linalool; campher; 1,8-cineol; lavandulol; lavandulyl acetate; 1-octen-3-ol; caryophyllene; farnesene; and 2-butyl-4,4,6-trimethyl-1,3-dioxan.

10. A perfumed article, comprising a fragrance substance composition as claimed in claim 4, wherein the article is selected from the group consisting of:
Perfume extracts, eau de parfums, eau de toilettes, after-shaves, eau de colognes, pre-shave products, splash colognes, perfumed refresher tissues, perfumes for acid, alkaline and neutral cleaning agents, washing agents, washing tablets, disinfectants as well as air improvers, aerosol sprays, waxes and polishes, as well as body care agents, bath oils, cosmetic emulsions, hair care products, deodorants and antiperspirants, products of decorative cosmetics as well as of candles, lamp oils, joss sticks, insecticides, repellents, and propellants.

11. A method for providing (a) hair or (b) textile fibres with one, two, three or all of the odor notes floral, rosy, rose oxide-like and herbal-like, preferably with a rose oxide-like and herbal-like odor note, comprising adding 2,4′-dimethylpropiophenone or a mixture containing 2,4′-dimethylpropiophenone.

12. A method of increasing the substantivity and/or retention of a fragrance substance composition comprising adding 2,4′-dimethylpropiophenone.

13. A method for producing a fragrance substance composition as claimed in claim 4, comprising the following step:
mixing 2,4′-dimethylpropiophenone with the or the further fragrance substances and optionally with further constituents of the fragrance substance composition.

14. The method as claimed in claim 13, wherein the 2,4′-dimethylpropiophenone is used in a quantity which is sufficient in the fragrance substance composition to impart, to modify and/or to reinforce one, two, three or all the odor notes floral, rosy, rose oxide-like and herbal-like.

15. The method as claimed in claim 2, wherein the odor notes are rose oxide-like and herbal-like.

16. The fragrance substance composition as claimed in claim 4, wherein the weight ratio of the total quantity of 2,4′-dimethylpropiophenone to the total quantity of further fragrance substances is in the range of 1:700 to 1:1.

17. The fragrance substance composition as claimed in claim 4, wherein the weight ratio of the total quantity of 2,4′-dimethylpropiophenone to the total quantity of further fragrance substances is in the range of 1:500 to 1:10.

18. The fragrance substance composition as claimed in claim 4, wherein the quantity of 2,4′-dimethylpropiophenone is sufficient to impart, to modify and/or to reinforce both of the odor notes rose oxide-like and herbal-like.

19. The fragrance substance composition as claimed in claim 4, wherein the fragrance substance composition comprises a quantity of 2,4′-dimethylpropiophenone in the range from about 0.1 to about 50% by weight, based on the total quantity of the fragrance substance composition.

20. The fragrance substance composition as claimed in claim 4, wherein the fragrance substance composition comprises a quantity of 2,4′-dimethylpropiophenone in the range from about 0.5 to about 30% by weight, based on the total quantity of the fragrance substance composition.

21. The fragrance substance composition as claimed in claim 4, wherein the fragrance substance composition comprises a quantity of 2,4′-dimethylpropiophenone in the range from about 0.5 to about 25% by weight, based on the total quantity of the fragrance substance composition.

22. The fragrance substance composition as claimed in claim 7, comprising 2, 3, 4, 5, 6, 7, 8 or more further fragrance substances.

23. The fragrance substance composition as claimed in claim 8, wherein the composition comprises 2, 3, 4 or more further rose fragrance substances.

24. The fragrance substance composition as claimed in claim 9, wherein the composition comprises 2, 3, 4 or more further herbal-herbaceous fragrance substances.

25. The method as claimed in claim 11, wherein the odor notes are a rose oxide-like or a herbal-like odor note.
